(19) United States
(12) Patent Application Publication
(10) Pub. No.: US 2005/0149101 A1
(43) Pub. Date: Jul. 7, 2005

(54) Non-traumatic surgical kit for uterine operations

(76) Inventor: Abdolhamid Huschmand Nia, Gottingen (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/503,244

(22) PCT Filed: Feb. 27, 2003
(86) PCT No.: PCT/DE03/00667

(30) Foreign Application Priority Data
Feb. 27, 2002 (DE) 102 08 508.0

(51) Int. Cl. A61M 29/00

(52) U.S. Cl. 606/193; 604/96.01; 600/116

(57) ABSTRACT

The invention relates to a non-traumatic surgical kit for uterine operations which comprises a manipulator and a pertubation rod in whose distal end two Luer adapters with one check valve each are provided. One of said Luer adapters is flow-connected via a supply line for air or saline solution with the interior of a balloon fastened on the proximal end of the manipulator and pertubation rod while a pertubation tube is connected to the other Luer adapter and centrally leads from the proximal end of the manipulator and pertubation rod extending through the interior of the balloon to an exit opening that is substantially flush with the proximal balloon wall. The surgical kit further comprises an outer tube concentrically enclosing the manipulator and pertubation rod at least across a partial length thereof. Said outer tube can be connected with its distal end with a suction device and with its proximal end with suction openings that are provided in a concave recess of a circular conical disk. Said conical disk is used for outer fixation is configured as an exchangeable template for a conization operation and can be axially displaced relative to the manipulator and pertubation rod.

The invention relates to a non-traumatic surgical kit for uterine operations, consisting of a manipulator and pertubation rod with a supply line for air or saline solution in fluidic communication with the interior of a balloon fastened on the proximal end of the manipulator and pertubation rod, and with a pertubation tube which opens out centrally via outlet opening from the proximal end of the manipulator and pertubation rod guided through the interior of the balloon.

Operations on the cervix uteri and some operations on the uterus, fallopian tubes, ovaries and uterine support apparatus necessitate maneuvering of the uterus, for example pulling, lifting and lowering movements, turning movements, securing and fixing. In vaginal operations, for example conization, in which a cone is excised from the tissue of the cervix uteri, the uterus is gripped with bullet forceps. Also, in order to determine the patency of the fallopian tubes, the uterus is manipulated with the aid of one instrument via the cervix uteri and with the aid of a second instrument through the abdominal wall, while the internal genitalia are inspected by endoscopy. Radiology or ultrasound examinations of the uterus and fallopian tubes with contrast agents also require manipulation of the cervix uteri.

It is an object of the invention to develop a surgical kit for uterine operations which offers versatile use while protecting the uterus.

According to the invention, this object is achieved by a non-traumatic surgical kit for uterine operations in which a bushing with an internal thread is guided on and axially displaceable relative to an external thread of the manipulator and pertubation rod, and a conical disk used for fixation forms the proximal end of the bushing.

The fact that the bushing provided with the conical disk is guided via a thread on the manipulator and pertubation rod permits extremely precise setting of the position of the conical disk. In addition, the conical disk can be pressed with great force on the os uteri externum by turning the bushing, which results in a better hold of the surgical kit and improved sealing. By means of the improved hold, it is now possible to move the uterus during the operation, for example upward into the abdominal cavity, without the surgical kit slipping or the uterus being perforated.

The surgical kit according to the invention also has the advantage that the uterus no longer has to be secured using sharp instruments.

In a departure from previously known designs in which the os uteri externum is sealed off, in the surgical kit according to the invention the os uteri internum is sealed off by the balloon and the os uteri externum is sealed off by the conical disk. Fluids can be instilled to check the patency of the fallopian tubes. Radiology and ultrasound contrast agents can be applied.

The kit according to the invention has a simple structure, is therefore inexpensive to produce, is easy to use, and can be sterilized.

According to the invention, the object is further achieved by a non-traumatic surgical kit for uterine operations in which the manipulator and pertubation rod can be connected via its distal end to a suction device and is connected via its proximal end to suction openings.

In this way, continuous suction in the wound bed is possible directly via the conical disk, particularly if a conization operation is being performed, i.e. in a surgical intervention in the cervix uteri in the area of the conical disk, or in vaginal hysterectomy (removal of the uterus through the vagina).

The suction openings can be designed, for example, as suction channels formed in the external thread of the manipulator and pertubation rod and/or the internal thread of the bushing. Upon production of the external thread, incisions need therefore simply be made in the longitudinal direction, which results in very economical production of the suction openings.

However, the suction openings can also be arranged in the bearing surface of the conical disk for the os uteri externum. Other designs of the suction openings and of the adjoining suction channels are also conceivable.

For a conization operation, the conical disk is expediently designed as a template, so that the cervix uteri can be circumscribed symmetrically and with millimeter precision.

The balloon, or its proximal wall, preferably forms the proximal end of the manipulator and pertubation rod and encloses the latter. Thus, in contrast to previously known designs, the rod no longer has to be used with an unprotected and solid point in the uterus. The balloon does not need to have any predetermined shape and it can be made of latex, whereas in the prior art use is made of a balloon made of rigid plastic and with a predetermined shape.

An outer tube is expediently provided which concentrically encloses the manipulator and pertubation rod along part of its length. The outer tube is preferably screwed with its proximal end into the distal end of the bushing. The bushing can also be integral with the outer tube.

To be able to approach the cervix uteri from all sides without complications, it is expedient if the manipulator and pertubation rod and outer tube are of rectilinear configuration. In this way, combinations of vaginal and abdominal operations are possible.

Gentle use of the kit is also ensured by the fact that the proximal portion of the manipulator and pertubation rod protruding from the outer tube or bushing and to be introduced into the cervix uteri has a smaller diameter than its distal rod portion.

It is particularly advantageous if the proximal portion of the manipulator and pertubation rod protruding from the outer tube or bushing and to be introduced into the cervix uteri is made flexible, for example by plastic deformability. In this way, the portion to be introduced into the cervix uteri can be adapted to the inclination of the uterus, while maintaining the axial displaceability of the bushing with conical disk relative to the manipulator and pertubation rod. It goes without saying that the portion to be introduced into the cervix uteri does not have to be flexible in its entirety, and instead, if appropriate, only some areas of it are flexibly deformable.

The conical disk is expediently designed to bear sealingly on the external os uteri of the cervix uteri. For this purpose, the conical disk can, for example, have a concave recess for bearing on the os uteri externum.

A scale extending in the longitudinal direction is preferably arranged in the area of the distal end of the bushing, on the outer wall of the manipulator and pertubation rod. In this way, the uterine probe length, that is to say the distance between the os uteri externum and the fundus uteri, can be determined in advance and the conical disk can if appropriate also be set to a defined probe length before the surgical kit is introduced.

It is particularly advantageous to provide a locking device, for example a scale with locking markings, for securely fixing the bushing on the manipulator and pertubation rod at defined positions. This makes it easier to set defined probe lengths during the operation and also provides for a further enhanced hold.

It is also advantageous if at least one of the adapters for the supply line and the pertubation tube in the area of the distal end of the manipulator and pertubation rod is a Luer adapter. At least one of the adapters can additionally be connected to a check valve or can be equipped with such a valve. In this way, the surgical kit can be connected easily and safely to standard instruments in the operating theater.

Illustrative embodiments of the invention are set out in the drawings, in which:

FIG. 1 shows a manipulator and pertubation rod whose proximal end is enclosed by a balloon;

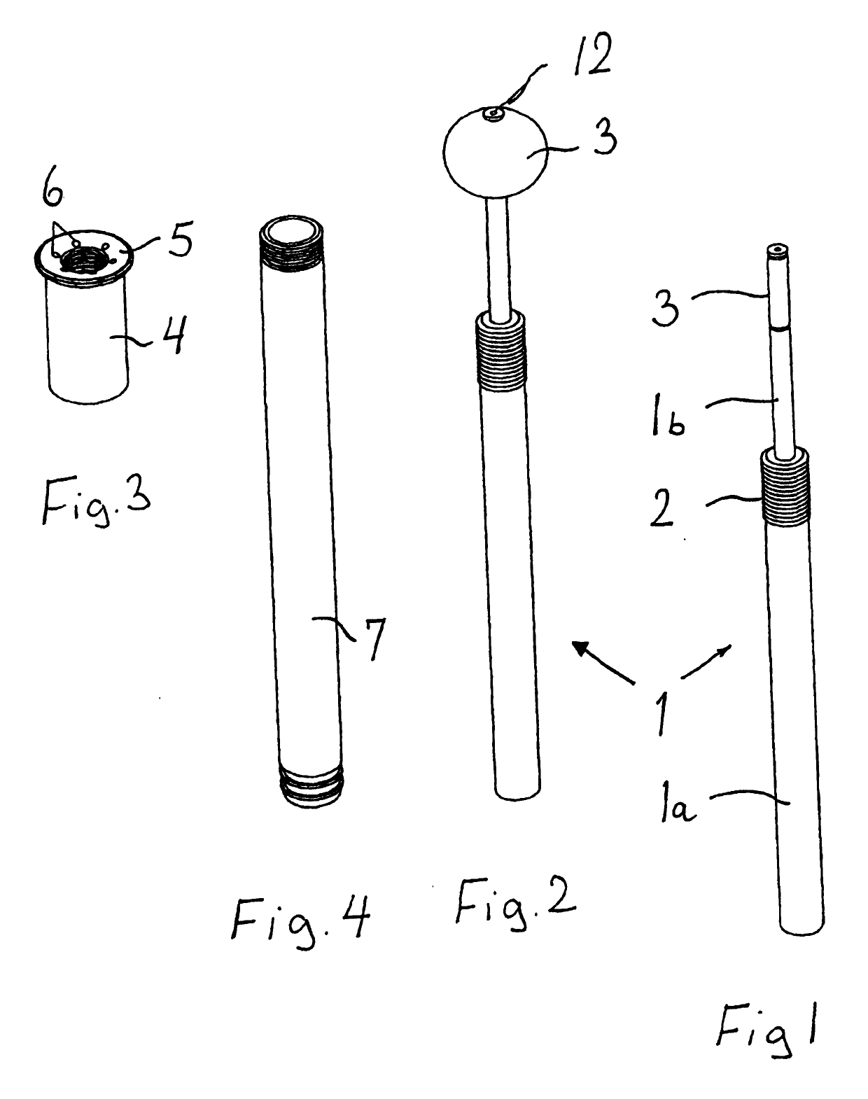

FIG. 2 shows the view according to FIG. 1, with the balloon inflated;

FIG. 3 shows a bushing equipped with a conical disk;

FIG. 4 shows an outer tube;

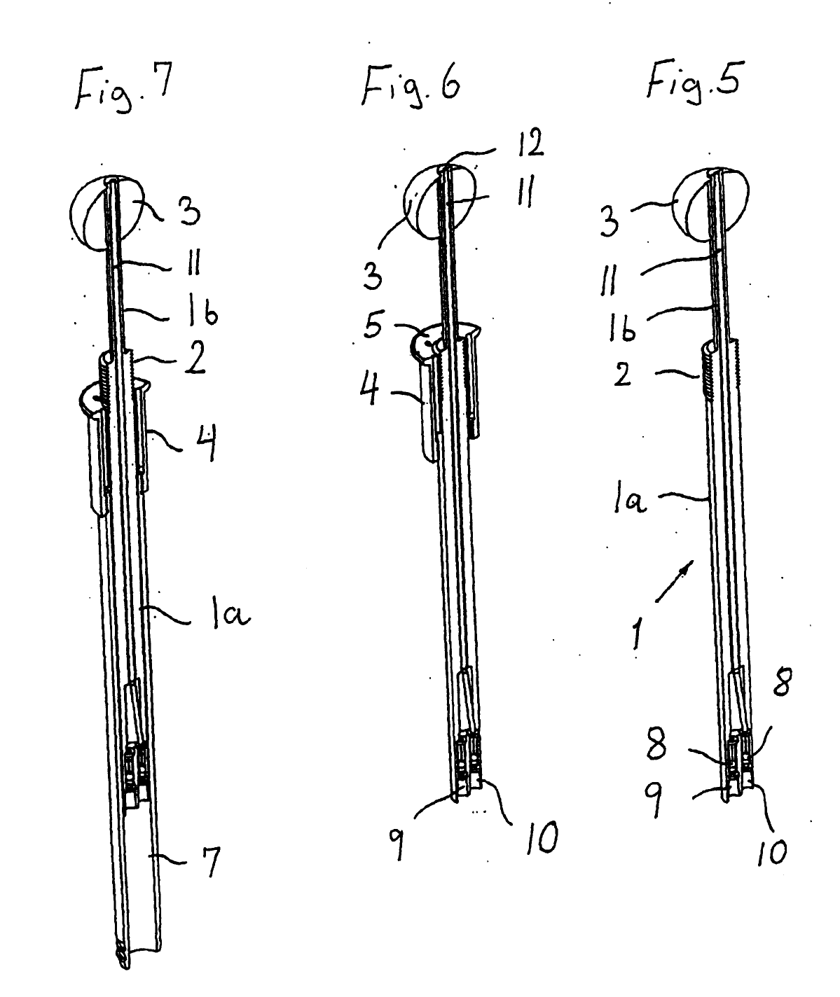

FIG. 5 shows a longitudinal section through the view according to FIG. 2;

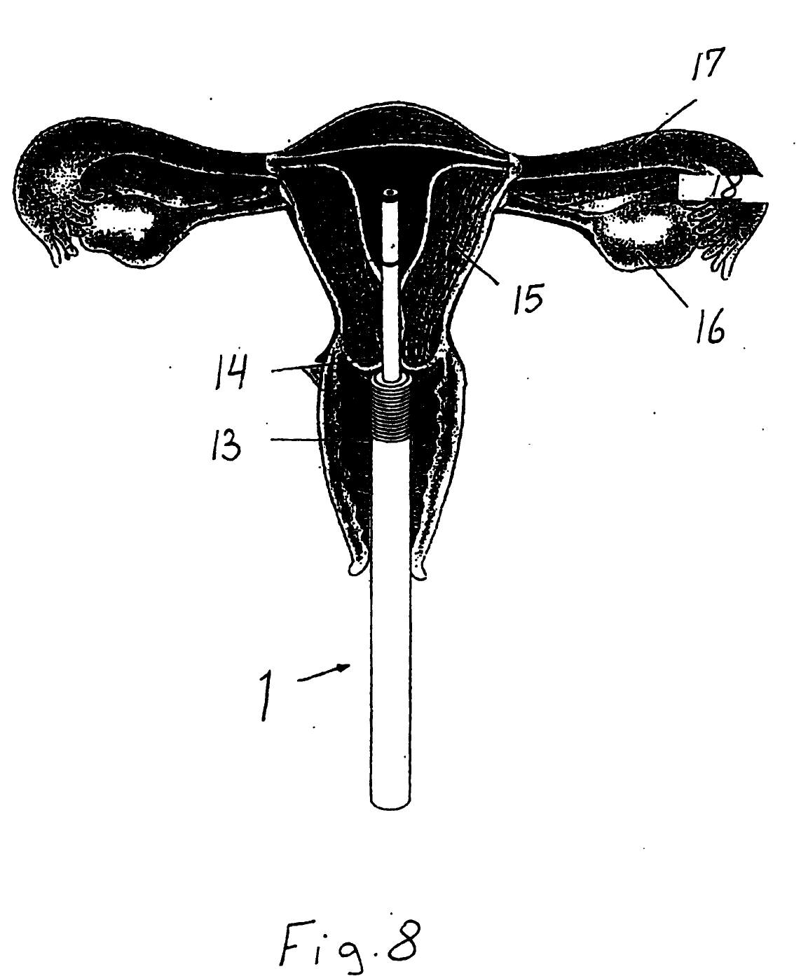

FIG. 6 shows the view according to FIG. 5, with the bushing according to FIG. 3 screwed on;

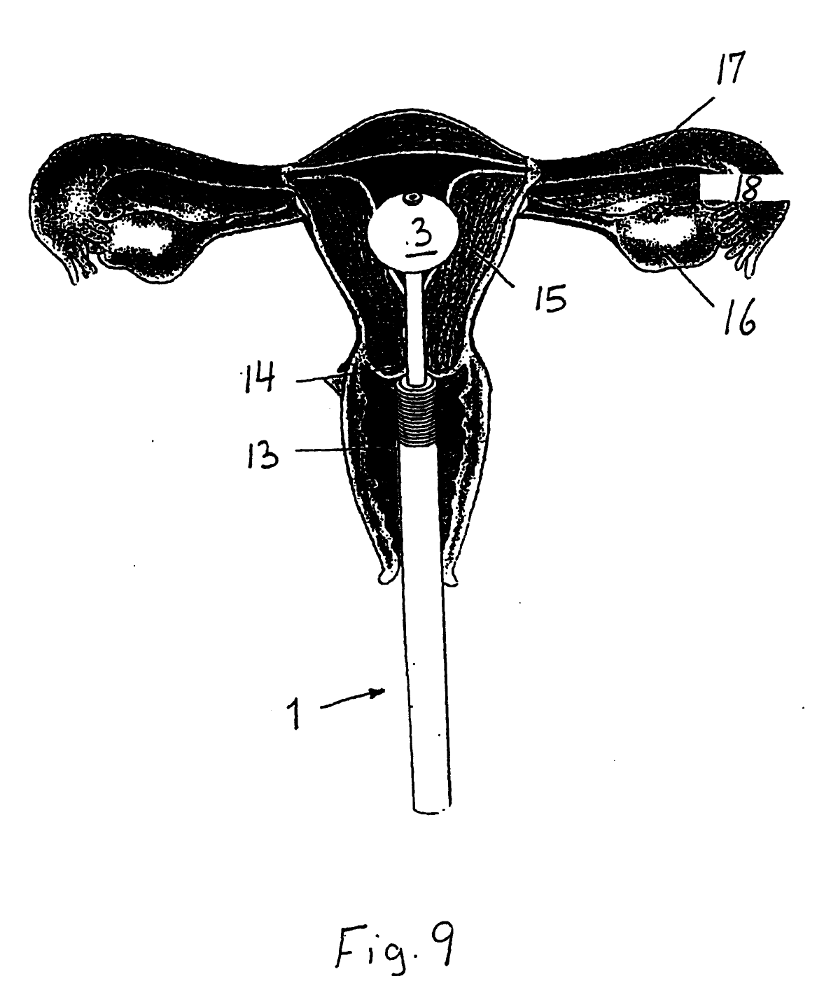

FIG. 7 shows the view according to FIG. 6, with the outer tube according to FIG. 4 screwed in;

FIG. 8 shows the manipulator and pertubation rod according to FIG. 1 introduced through the vagina and cervix uteri into the uterus;

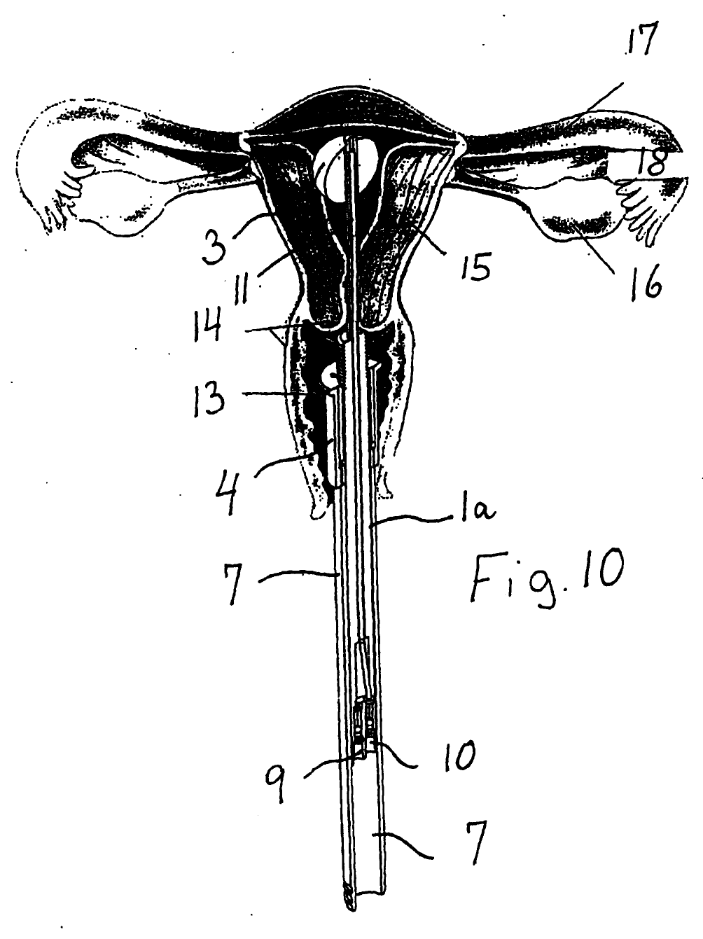

FIG. 9 shows a view according to FIG. 8, with the balloon according to FIG. 2 filled;

FIG. 10 shows a view according to FIG. 8, with the suction device according to FIG. 7 applied;

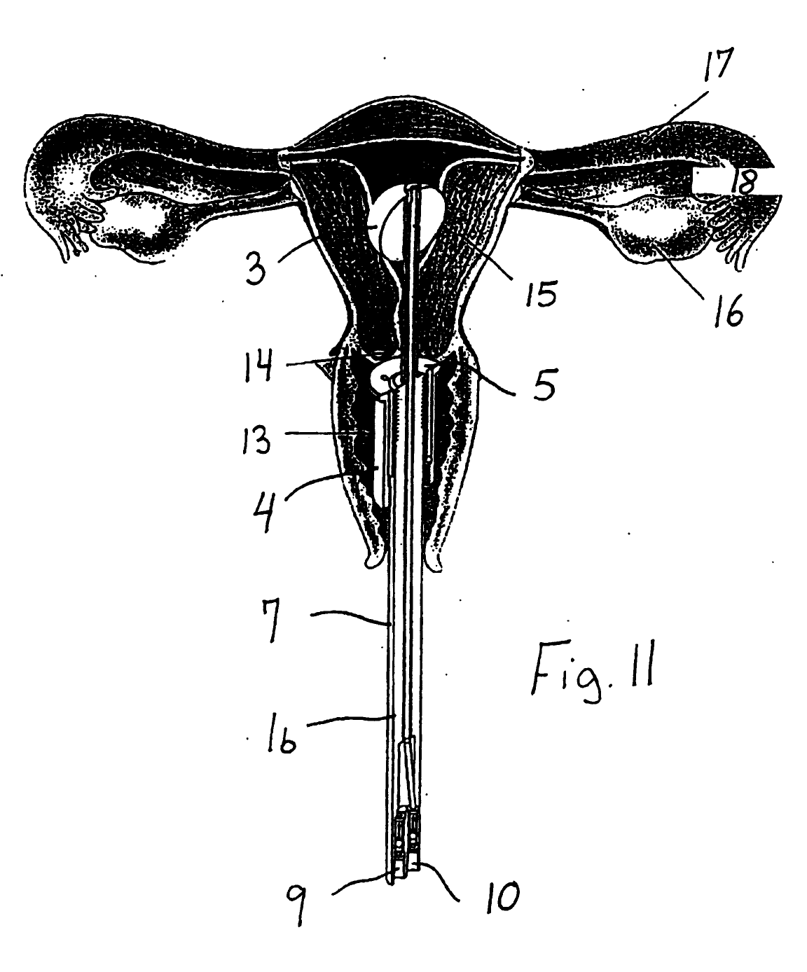

FIG. 11 shows, in a view according to FIG. 8, the surgical kit for uterine operations according to FIG. 10, with the bushing according to FIG. 3 pushed axially up and bearing on the cervix uteri;

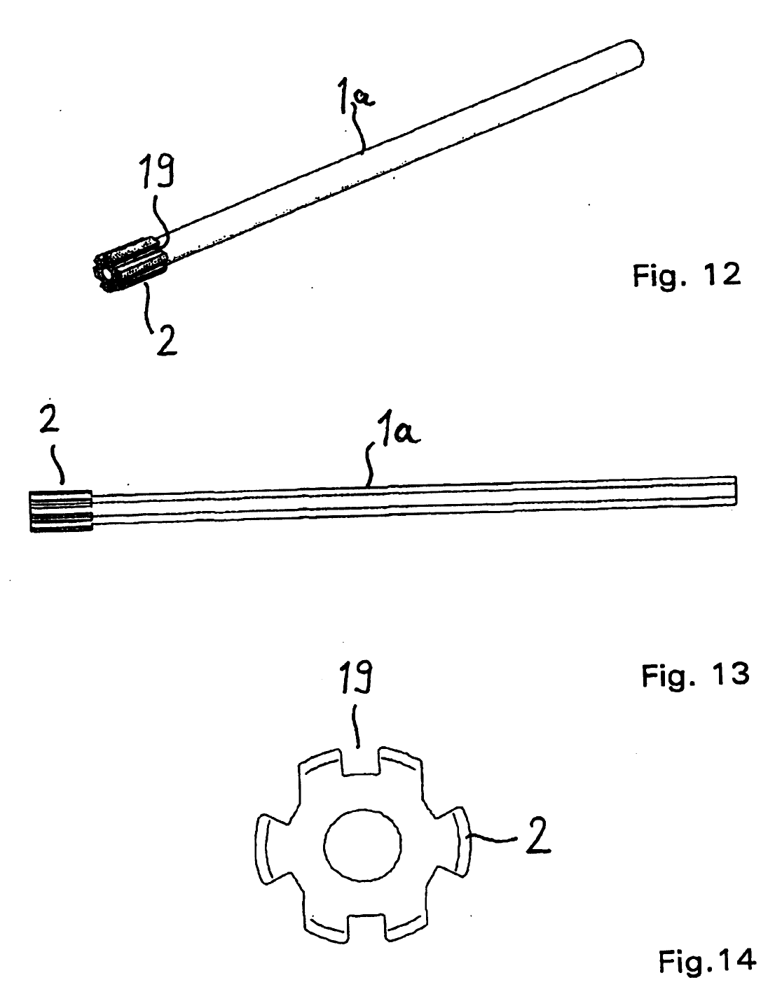

FIG. 12 shows a perspective view of a second embodiment of the distal portion 1a of the manipulator and pertubation rod with external thread and suction channels in the external thread;

FIG. 13 shows a side view of the distal portion of the manipulator and pertubation rod from FIG. 12; and

FIG. 14 shows a front view of the external thread of the distal portion of the manipulator and pertubation rod according to FIGS. 12 and 13.

FIG. 1 shows a manipulator and pertubation rod 1 whose distal portion la of greater diameter is provided with an external thread 2 in its upper area, and whose proximal portion 1b of smaller diameter is enclosed by a balloon 3, which is shown in the inflated or filled state in FIG. 2.

FIG. 3 shows a bushing 4 which can be screwed onto the external thread 2 of the rod 1 and whose proximal end is formed by a conical disk 5 in which suction openings 6 are provided which can be connected via an outer tube 7, shown in FIG. 4, to a suction device (not shown). These suction openings 6 are provided in a concave recess of the circular conical disk used for outer fixation, which can be designed as an exchangeable template for a conization operation.

FIG. 5 shows that in the distal end of the manipulator and pertubation rod 1 there are two Luer adapters 9, 10 which are each provided with a check valve 8 and of which one adapter 9 is in fluidic communication with the interior of the balloon 3 via a supply line for air or saline solution, while a pertubation tube 11 is connected to the other Luer adapter 10 and opens out centrally from the proximal end of the rod 1, guided through the interior of the balloon 3, via an outlet opening 12 which lies substantially flush in the proximal balloon wall (see also FIG. 2).

FIG. 6 shows the bushing 4 screwed onto the external thread 2 of the rod 1. According to FIG. 7, the proximal end of the outer tube 7 shown in FIG. 4, and pushed onto the distal portion la of the rod 1, is screwed into the lower end of the bushing 4, which outer tube 7 can be connected via its distal end to a suction device, and is connected to the suction openings 6 via its proximal end, screwed into the bushing 4.

FIG. 8 shows the manipulator and pertubation rod 1 which is introduced into the vagina 13 and whose distal portion 1a of smaller diameter is guided through the cervix uteri 14 and whose end enclosed by the balloon 3 protrudes into the uterus 15. The figure shows ovary 16, fallopian tube 17 and fimbria 18.

FIG. 9 corresponds to FIG. 8 and simply shows the inflated or filled balloon 3.

FIG. 10 shows the manipulator and pertubation rod 1 according to FIG. 9, but now with the bushing 4 which carries the outer tube 7, is screwed onto the external thread 2 of the rod 1, and forms part of the suction device, and which bushing 4, according to FIG. 11, is pushed axially upward, by turning the outer tube 7 on the external thread 2, to such an extent that the conical disk 5 lies with support on the cervix uteri 14.

FIG. 12 shows an embodiment of the manipulator and pertubation rod 1 with an external thread 2 into which longitudinally extending suction channels 19 are let in. The suction channels can either be milled or can already be provided in the mold used in injection-molding.

The suction channels 19 open out in the area of the conical disk of the bushing 4 screwed onto the external thread 2, so that suction can take place in the operating area.

FIG. 13 shows the embodiment of the manipulator and pertubation rod 1 from FIG. 12 in cross section. It is clear from the figures that several suction channels 19 are distributed about the circumference, preferably at regular intervals.

The arrangement of the suction channels 19 in the external thread 2 is clearer from FIG. 14 which shows a front view of the proximal end of the manipulator and pertubation rod 1. It can be seen that the suction channels 19 do not all need to have the same cross section. Instead, for manufacturing reasons, it is expedient if the suction channels 19 lying to the side in an injection mold are not designed approximately rectangular, and instead, in addition, a recess is provided extending in the direction of travel of the injection mold.

The surgical kit or the manipulator and pertubation rod should be made of radiopaque material.

1. A non-traumatic surgical kit for uterine operations, with a manipulator and pertubation rod, with a supply line for air or saline solution in fluidic communication with the interior of a balloon fastened on the proximal end of the manipulator and pertubation rod, and with a pertubation tube which opens out centrally via an outlet opening from the proximal end of the manipulator and pertubation rod guided through the interior of the balloon, characterized in that a bushing with an internal thread is guided on and axially displaceable relative to an external thread of the manipulator and pertubation rod, and a conical disk used for fixation forms the proximal end of the bushing.

2. A non-traumatic surgical kit for uterine operations, consisting of a manipulator and pertubation rod, with a supply line for air or saline solution in fluidic communication with the interior of a balloon fastened on the proximal end of the manipulator and pertubation rod, and with a pertubation tube which opens out centrally via an outlet opening from the proximal end of the manipulator and pertubation rod guided through the interior of the balloon, characterized in that the manipulator and pertubation rod can be connected via its distal end to a suction device and, in the area of its proximal end, it is connected to suction openings.

3. The surgical kit as claimed in claim 2, characterized in that the suction openings are designed as suction channels formed in the external thread of the manipulator and pertubation rod and/or the internal thread of the bushing.

4. The surgical kit as claimed in claim 2, characterized in that the suction openings are arranged in the bearing surface of the conical disk for the os uteri externum

5. The surgical kit as claimed in claim 2, characterized in that a bushing with an internal thread is guided on and axially displaceable relative to an external thread of the manipulator and pertubation rod, and a conical disk used for fixation forms the proximal end of the bushing.

6. The surgical kit as claimed in claim 1, characterized by an outer tube concentrically enclosing the manipulator and pertubation rod at least along part of the length thereof.

7. The surgical kit as claimed in claim 6, characterized in that the outer tube is screwed with its proximal end into the distal end of the bushing.

8. The surgical kit as claimed in claim 1, characterized in that manipulator and pertubation rod and outer tube are of rectilinear configuration.

9. The surgical kit as claimed in claim 1, characterized in that the proximal portion of the manipulator and pertubation rod protruding from the outer tube or bushing and to be introduced through the cervix uteri into the uterus has a smaller diameter than its distal rod portion.

10. The surgical kit as claimed in claim 1, characterized in that the proximal portion of the manipulator and pertubation rod protruding from the outer tube or bushing and to be introduced through the cervix uteri is flexible at least in some areas.

11. The surgical kit as claimed in claim 1, characterized in that the conical disk is designed as a template for a conization operation.

12. The surgical kit as claimed in claim 1, characterized in that the conical disk is designed to bear sealingly on the os uteri externum of the cervix uteri.

13. The surgical kit as claimed in claim 1, characterized in that the conical disk has a concave recess.

14. The surgical kit as claimed in claim 1, characterized in that a scale extending in the longitudinal direction is arranged at least in the area of the distal end of the bushing, on the outer wall of the manipulator and pertubation rod.

15. The surgical kit as claimed in claim 1, characterized in that a locking device is provided for securely fixing the bushing on the manipulator and pertubation rod at defined positions.

16. The surgical kit as claimed in claim 1, characterized in that at least one of the adapters of the supply line or of the pertubation tube in the area of the distal end of the manipulator and pertubation rod is a Leer adapter.

17. The surgical kit as claimed in claim 1, characterized in that at least one of the adapters of the supply line or of the pertubation tube is connected to or equipped with a check valve.

18. The surgical kit as claimed in claim 1, characterized in that the outlet opening of the pertubation tube lies substantially flush in the proximal balloon wall.
